(19) United States
(12) Patent Application Publication
(10) Pub. No.: US 2014/0214003 A1
(43) Pub. Date: Jul. 31, 2014

(54) Guidewire and Catheter System and Method for Treating a Blood Clot

(71) Applicants: Yi Yang, San Francisco, CA (US); Scott L. Jahrmarkt, Miami Beach, FL (US); Michele Migliuolo, Pittsburgh, PA (US); Juan Carlos Diaz, Pembroke Pines, FL (US)

(72) Inventors: Yi Yang, San Francisco, CA (US); Scott L. Jahrmarkt, Miami Beach, FL (US); Michele Migliuolo, Pittsburgh, PA (US); Juan Carlos Diaz, Pembroke Pines, FL (US)

(21) Appl. No.: 14/111,051

(22) PCT Filed: Jul. 14, 2011
(86) PCT No.: PCT/US11/43984

§ 371 Date: Oct. 10, 2013

Related U.S. Application Data
(60) Provisional application No. 61/365,147, filed on Jul. 16, 2010.

(51) Int. Cl. A61M 25/10; A61M 25/09; A61M 1/00

(52) U.S. Cl. CPC A61M 25/1025; A61M 25/10184; A61M 1/008; A61M 25/09
USPC 604/509; 604/103.01; 604/99.01

(57) ABSTRACT

A system for treating a blood clot includes a microcatheter extending through an optional aspiration catheter, and a guidewire subassembly extending through and beyond the microcatheter, the guidewire assembly having a guidewire, an element coupled to and extending around a portion of the guidewire, and a balloon coupled to and extending around the element. The balloon includes or is coupled to a proximal seal in contact with the inner surface of the microcatheter and has at least one radially arranged pore. Infusate flows through the microcatheter into the element and between the element and the guidewire, and out and into the balloon to pressurize the balloon. The infusate escapes the balloon through the pores.

### BACKGROUND OF THE INVENTION

1. Field of the Invention

This invention relates broadly to systems and methods for treating blood clots in patients. More particularly, this invention relates to systems and methods for treating a blood clot in the brain of a patient.

2. State of the Art

A stroke is caused by a rupture or an occlusion of a blood vessel which leads to oxygen deprivation in the brain. In the United States, nearly eight hundred thousand people suffer a stroke each year, and over one hundred and forty thousand people die from strokes each year. Stroke is the leading cause of serious, long-term disability in the United States and the third leading cause of death. Approximately three-quarters of strokes in the United States are first attacks and approximately one-quarter are recurrent attacks. Eighty seven percent are ischemic in nature, meaning that they are caused by a restriction, obstruction, or blockage in the blood supply of the patient, and thirteen percent are hemorrhagic, meaning that they are caused by excessive bleeding. The economic cost of stroke to the United States is over forty billion dollars per year. The direct costs of medical care and therapy are almost thirty billion dollars per year.

It is well known in the art that the extent to which treatment of a stroke is successful in preventing death and/or in reducing the consequent damage to a patient is largely influenced by the time which elapses between the onset of the stroke and the proper treatment of the stroke. The elapsed time is a function of not only whether or not a patient is able to get to a medical facility or hospital, but also the nature of the stroke and whether or not the particular medical facility or hospital to which the patient is initially brought is best equipped to treat the stroke. The capability of the medical facility to treat the particular stroke may not be known until the patient is properly evaluated and analyzed. Generally, if more than three hours elapse between the onset of the stroke and treatment, then a combination of tPA (Tissue Plasminogen Activator—a drug used to dissolve blood clots) and mechanical treatments need to be utilized.

If a cerebral clot is diagnosed and removed within four hours of the clot's formation, a patient generally has a better chance to recover fully. If a neurointerventionist happens to be present (most are generally located at stroke centers), then certain devices may be available to remove the cerebral clot. One device is the Merci retrieval device made by Concentric Medical. With the Merci device, a small catheter (e.g., having a 0.015″ inner diameter) is advanced through the femoral artery and fed up to the brain. A special Nitinol wire is advanced through the catheter to the clot. The wire changes form after passing through the clot and can be used to pull out the clot. A second device, sold by Penumbra, Inc. also uses a small catheter which is advanced through the femoral artery and fed up to the brain, but instead of pulling the clot out mechanically, utilizes suction to pull out the clot. Both of these devices are often unsuccessful in their intended functions.

### SUMMARY OF THE INVENTION

The invention provides a system and method for treating a blood clot in the brain of a patient. The system includes a catheter/guidewire assembly adapted to be inserted in the artery system of the patient. The catheter/guidewire assembly includes an optional aspiration catheter, a microcatheter insertable through the aspiration catheter when provided, and a guidewire subassembly. The guidewire subassembly includes a guidewire which extends through the microcatheter, a support element which is affixed to the guidewire, and a weeping or microjet balloon (i.e., a balloon with one or more small holes) which is affixed to the outside of the support element.

In one embodiment the support element includes a proximal tubular section which is affixed to the guidewire, a first helical (coiled) section which is loose around the guidewire, a second tubular section which supports the proximal end of the balloon and is loose around the guidewire, a second helical section which extends through the balloon, and a distal third tubular section which is also affixed to the guidewire and to which the distal end of the balloon can be attached. The proximal end of the balloon preferably includes a flared portion which contacts the inner wall of the microcatheter. With the guidewire subassembly arranged in this manner, infusate which is injected through the microcatheter is prevented from exiting the distal end of the microcatheter by the flared portion of the balloon and will instead enter the support element at its first helical section. From there, the infusate will flow between the guidewire and the support element and out of the support element at its second helical section and into the balloon. The infusate will inflate the balloon, and when the infusate pressure reaches a desired level, the infusate will weep through the pores of the balloon.

In one embodiment, a cage element is provided around the balloon. The proximal end of the cage element may be attached to the balloon where the balloon attaches to the support element. Alternatively, the proximal end of the cage element may be attached to the distal end of the microcatheter. In one embodiment, the distal end of the cage element is attached to either the distal end of the support element or to the guidewire or may be attached to the balloon where the balloon attaches to the support element. In another embodiment, the distal end of the cage element is unattached to the catheter/guidewire assembly. According to one aspect of the invention, the cage element is arranged to restrain expansion of the balloon. According to another aspect of the invention, the cage element is arranged to remain open after balloon inflation in order to keep the clot open and allow blood to flow to the vessels that were affected by the clot. In this sense, the cage acts as a removable stent.

In one embodiment the catheter/guidewire assembly is a relatively short assembly and is intended for insertion through the carotid artery. In another embodiment the catheter/guidewire assembly is a relatively longer assembly and is intended for insertion through the femoral artery.

The assembly may be used as follows. First, either the femoral or carotid artery is punctured and a sheath inserted. A steerable guidewire is inserted into the sheath and steered until it crosses the clot of interest. The sheath is then removed, and the aspiration catheter of the described system is inserted through the puncture over the guidewire and up to just proximal the clot. The microcatheter of the described system is then fed between the aspiration catheter and the guidewire until it extends out of the aspiration catheter and into the clot. The steerable guidewire is then removed, and the guidewire subassembly of the described system with the guidewire, attached support element and balloon are inserted into the microcatheter until the balloon is located in the clot (with the distal end of the guidewire typically extending past the clot). Alternatively, the guidewire subassembly may be used initially to function in place of the steerable guidewire, thereby eliminating the need for the steerable guidewire and reducing the number of insertion steps. Infusate (e.g., tPA alone or in combination with a radiopaque constrast agent) is then injected into the microcatheter, enters the support element at its first helical section, flows between the guidewire and the support element and out of the support element at its second helical section and into the balloon. Sufficient pressure is applied to the infusate to inflate the balloon and cause the infusate to either weep or jet out of the pores of the balloon (depending upon force applied to the infusate) and into the clot or into the walls of the blood vessel. With a contrast agent, the expansion of the balloon and the flow of the infusate within the occluded vessel can be monitored in real-time. When sufficient infusate has been introduced into the clot or vessel walls, the pressure is removed, the balloon deflates, and the microcatheter and guidewire subassembly are removed from the aspiration catheter. It is anticipated that the tPA in the infusate may completely lyse and dissolve the clot to effect recanalization, rendering subsequent aspiration of the clot unnecessary. However, if necessary suction may then be applied to the aspiration catheter in order to remove the clot. The aspiration catheter is then removed and the artery is closed.

There are several methods currently being used by physicians for intravascular treatments that can be used in conjunction with the microcatheter/guidewire of the invention to effect re-canalization. A guiding catheter can be used as an initial support for the microcatheter/guidewire. If the guiding catheter cannot get close enough to the clot, another “aspiration catheter” is used, which is more flexible and able to track more distal. Then the microcatheter/guidewire is inserted. Also, physicians can group the aspiration catheter and microcatheter/guidewire devices together as a system and insert the system up to the vasculature. The microcatheter/guidewire is then fed to the clot. In all methods, aspiration, when performed, is preferably performed through the catheter that is closest to the clot.

Objects and advantages of the invention will become apparent to those skilled in the art upon reference to the detailed description taken in conjunction with the provided figures.

### BRIEF DESCRIPTION OF THE DRAWINGS

FIG. 1 is a transparent perspective view of a first embodiment of the invention.

FIG. 1a is a broken, transparent side view of the embodiment of FIG. 1.

FIG. 1b is a transparent perspective view of the embodiment of FIG. 1 in an inflated position.

FIG. 1c is broken, transparent side view of FIG. 1b.

FIG. 1d is a partially transparent side view and partially cross-sectional view of a portion of the embodiment of FIG. 1 without the balloon.

FIG. 1e is a partially transparent side view and partially cross-sectional view of the same portion of the embodiment shown in FIG. 1d but with the balloon and without the core wire.

FIG. 2 is a transparent perspective view of a second embodiment of the invention.

FIG. 2a is a broken, transparent side view of the embodiment of FIG. 2.

FIG. 2b is a transparent perspective view of the embodiment of FIG. 2 in an inflated position.

FIG. 2c is a broken, transparent side view of FIG. 2b.

FIG. 2d is a broken, transparent side view of the embodiment of FIG. 2 with the cage expanded and the balloon collapsed.

FIG. 3 is a transparent perspective view of a third embodiment of the invention.

FIG. 3a is a broken, transparent side view of the embodiment of FIG. 3.

FIG. 3b is a broken, transparent side view of the embodiment of FIG. 3 in an inflated position.

FIG. 3c is a broken, transparent side view of the embodiment of FIG. 3 with the cage expanded and the balloon collapsed.

FIG. 4 is a broken side view of a distal portion of the third embodiment of the invention, showing an alternative cage and balloon construction.

FIG. 5 is a broken side view of a distal portion of the third embodiment of the invention, showing another alternative cage and balloon construction.

FIG. 6 is a schematic longitudinal section view of a fourth embodiment of the invention.

FIG. 7 is a broken, partial perspective and partial transparent side view of a fifth embodiment of the invention.

FIG. 8 is a schematic longitudinal section view of a sixth embodiment of the invention.

FIGS. 8a and 8b are enlarged views of portions of FIG. 8.

### DETAILED DESCRIPTION OF THE PREFERRED EMBODIMENTS

For purposes of this disclosure, the terms ‘proximal’ and ‘distal’ are referenced relative to the hand of the operator of the guidewire and catheter system when the system is in use, as well as the site at which the system is inserted into the patient's body; system components and anatomical structure closer to the operator's hand and insertion site are considered relatively ‘proximal’, whereas system components and anatomical structure further from the operator hand and insertion site are considered relatively ‘distal’.

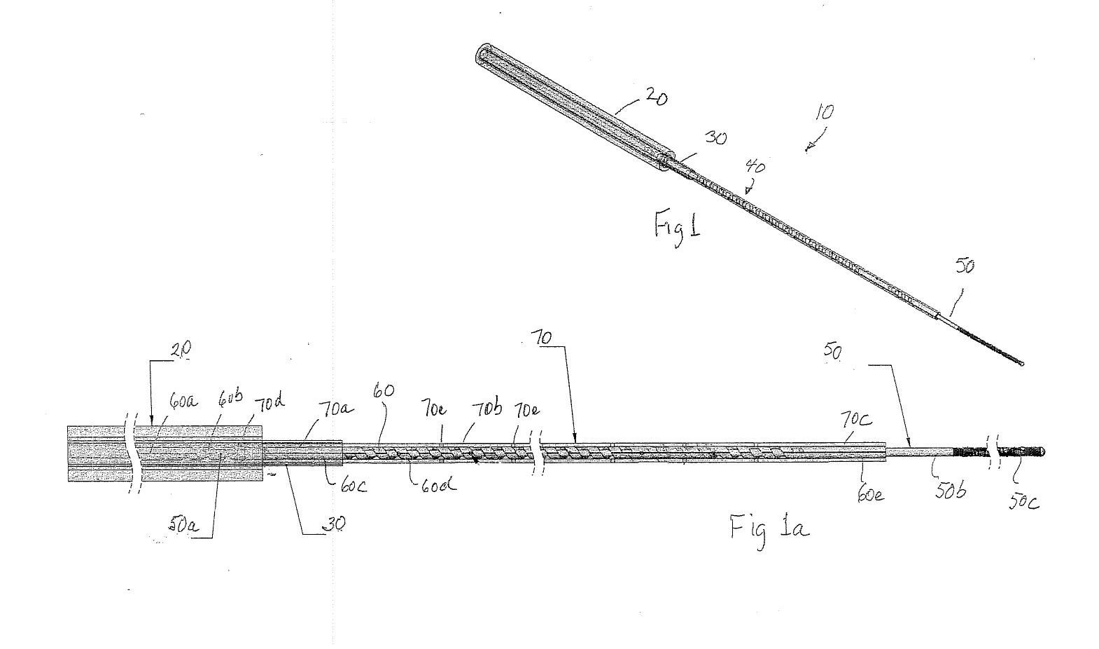

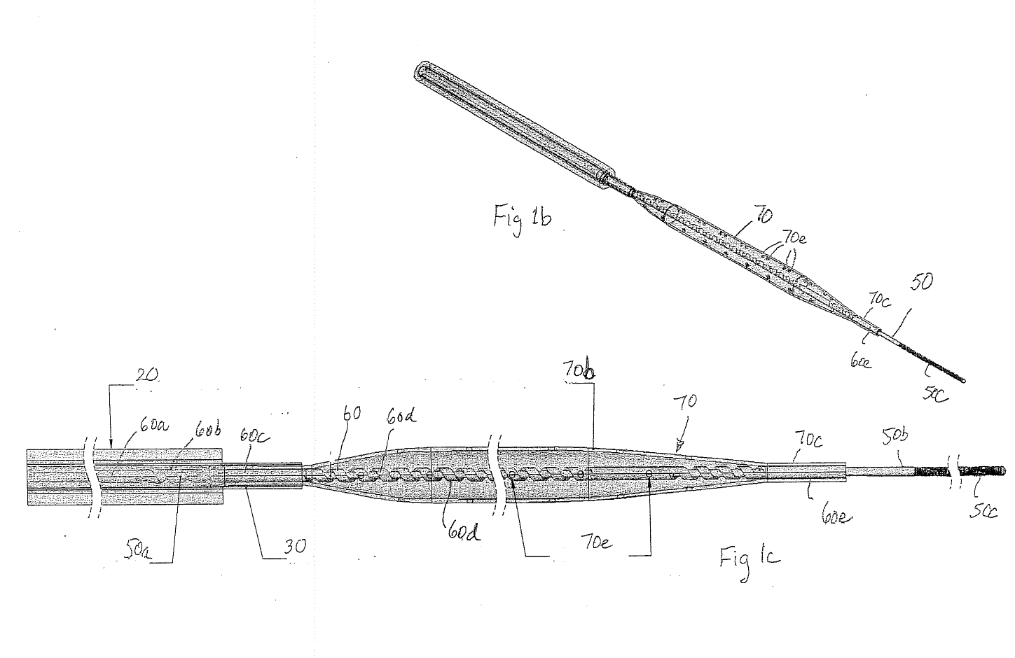

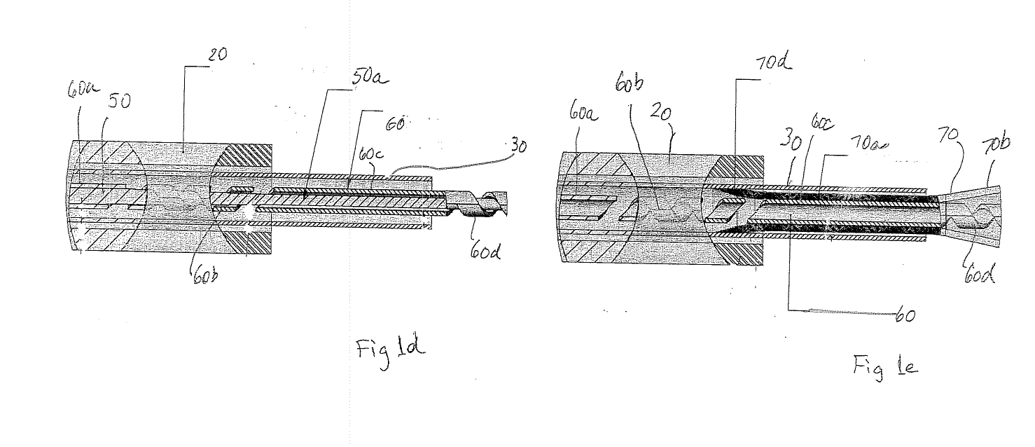

Turning to FIG. 1 and FIGS. 1a-e, a first embodiment of a catheter/guidewire system 10 is seen for treating blood clots in the intracranial vasculature of a patient. System 10 includes an aspiration catheter 20, a microcatheter 30, and a guidewire subassembly 40. Aspiration catheter 20, which may be a guiding catheter or other support catheter, is preferably approximately 120 cm-160 cm in length if it is to be introduced through the femoral artery, or approximately 20 cm-40 cm in length if it is to be introduced through the carotid artery, and has an inner diameter of between 0.040 and 0.060 inches and an outer diameter of between 0.06 and 0.10 inches. The microcatheter 30 is preferably slightly longer than the aspiration catheter 20 and insertable through the aspiration catheter. The microcatheter 30 preferably has an inner diameter of between 0.020 and 0.030 inches and an outer diameter of 0.023-0.033, with a wall thickness of approximately 0.003 inches. The microcatheter is preferably formed from a plastic extrusion with a stainless steel coil or braid. The guidewire subassembly 40 is preferably slightly longer than the microcatheter 30 and insertable through the microcatheter. The guidewire subassembly 40 includes a guidewire 50, a support element 60, and a weeping or microjet balloon 70. As will be described in more detail hereinafter, a proximal end 60a of the support element 60 attaches to the guidewire 50.

Guidewire 50 preferably has a diameter of between 0.012 and 0.018 inches along most of its length. As is seen best in FIG. 1d, just distal the point of attachment of the support element 60 to the guidewire 50, the guidewire portion 50a has a decreased diameter in order to permit infusate flow between the guidewire portion 50a and the support element 60 as described hereinafter. The very distal end 50b of the guidewire also preferably decreases in diameter down to approximately 0.004-0.006 inches in diameter and terminates in a coil 50c. The distal end of the guidewire 50b may or may not be exposed. In other words, the coil 50c may butt up against the distal end 60e of the support element 60 such that the tip of the guidewire assembly has the same diameter as the proximal end of the guidewire, with only a coil exposed. The guidewire is preferably formed from stainless steel, Nitinol, or from another very flexible material.

The support element 60 of the guidewire subassembly is preferably a thin tube having helical cut-outs formed or cut in large portions thereof. Alternatively, the support element is formed from a helical coil having open wound portions. More particularly, support element preferably includes a small proximal affixation portion 60a having an inner diameter that is substantially equal to or less than the outer diameter of the main length of the guidewire core wire 50. The proximal end 70a of the balloon (balloon neck) sits on top of the support element. Preferably, though not required, the combined diameter of the proximal fixation portion 60a of the support element and the thickness of the balloon neck does not exceed the diameter of the main length of guidewire. The affixation portion 60a is preferably tubular (although it could have holes and could be helical) and is affixed to the guidewire by soldering, brazing, welding, gluing, or other fixing techniques known in the art. Distal the affixation portion 60a of the support element is a first helical or coil portion 60b which loosely surrounds the decreased diameter portion 50a of the guidewire, thereby permitting infusate to enter the support element and to flow between it and the guidewire portion 50a. The coil portion 60b terminates in a small balloon support portion 60c which is preferably tubular and to which a proximal end portion (balloon neck) 70a of balloon 70 is attached by glue or by other well-known techniques to its outside surface. In this manner, support element 60 runs inside balloon 70. Distal the support section 60c, support element 60 has a second helical or coil portion 60d which is located inside the balloon 70, thereby permitting infusate to exit the support element and enter the balloon 70. As seen in FIG. 1c, the second helical or coil portion 60d of support element 60 can extend entirely through the balloon 70, or can (less preferably) include a closed wall tubular construct as one or more portions of the second portion 60d. Regardless, the distal end of the support element may include a preferably small tubular support portion 60e to which the distal end 70b of balloon 70 is attached, thereby preventing infusate from exiting from between the balloon 70 and the support element 60. Tubular support portion 60e is attached to the guidewire 50 to likewise prevent infusate from exiting the support element 60. The support element is preferably formed from Nitinol or stainless steel. If desired, portions 60a and 60e of the support element 60 can be made of or coated with a material such as platinum-iridium which under fluoroscopy can be used to help locate the position of the guidewire assembly. Because the large majority of support element 60 is preferably cut as a helix or coil, support element 60 is very flexible and does not affect the flexibility of the portion of the guide wire 50 extending within the balloon.

Balloon 70 has a proximal portion 70a attached to tubular support portion 60a of support element 60, a distal portion 70b attached to tubular support portion 60e of the support element 60, and an expandable middle portion 70c which extends around support element 60. The proximal portion 70a includes a proximal seal 70d (seen best in FIG. 1e) which is not directly attached to the support element 60 and which flares out to an outer diameter slightly larger than the inner diameter of the microcatheter 30 such that the seal 70d is always under compression by the inner surface of the microcatheter and prevents infusate from exiting the distal end of the microcatheter. The expandable middle portion 70c of the balloon 70 preferably includes one or more microholes or pores 70e, each preferably not exceeding a diameter of 0.002 inch, which permit infusate to escape out of the balloon when sufficient pressure is applied (e.g., preferably less than 760 Torr above blood pressure and more preferably less than 400 Torr above blood pressure). The balloon may be a weeping balloon, in which the microholes or pores of sufficient dimension and/or number, and wherein appropriate pressure is applied to the infusate, to cause the infusate to weep or seep out of the balloon in a low pressure manner. Alternatively, the balloon may be a microjet balloon, with holes (or micropores) of size and number (e.g., one or more micropores) such that the infusate jets out of the balloon when the balloon is pressurized toward or in an expanded configuration. When a microjet balloon is employed, the clot is agitated by the force of the infusate jet to accelerate dissolution of the clot. A flow rate of 0.1 cc/sec of infusate through a balloon with two micropores (as shown in FIG. 4 described hereinafter) has been shown to be effective for achieving microjetting of the infusate, desired clot agitation, and clot dissolution. The balloon 70 may be made from silicon, polyurethane, latex, Kraton™ polymers (i.e., styrenic block copolymers consisting of polystyrene blocks and rubber blocks), or other materials suitable for use in a low pressure compliant balloon. Typically, the balloon is between 0.001 and 0.008 inches thick, between 0.4 and 0.8 inches long and is capable of having a nominal expanded diameter of no more than 0.18 inches. Balloon lengths will typically range from 0.2 up to 2 inches in length. Balloon 70 is seen in an inflated (expanded) state in FIGS. 1b and 1c and in a deflated (unexpanded) state in FIGS. 1 and 1a.

The guidewire/catheter system 10 may be used as follows. First, either the femoral or carotid artery (not shown) is punctured and a sheath (not shown) inserted. A steerable guidewire (not shown) is inserted into the sheath and steered until it crosses the clot of interest (not shown). The sheath is optionally then removed, and the aspiration catheter 20 is then inserted through the sheath (if still present) and the puncture and over the guidewire and up to just proximal the clot (i.e., preferably not inserted through the clot). The microcatheter 30 is then fed between the aspiration catheter 20 and the guidewire until it extends out of the aspiration catheter and into the clot. The steerable guidewire is then removed, and the guidewire subassembly 40 with the guidewire 50, attached support element 60 and balloon 70 are inserted into the microcatheter 30 and snaked (extended) through the microcatheter until the balloon 70 is located within the clot (with the distal end 50b of the guidewire typically extending past the clot). Alternatively, the guidewire subassembly may be used initially to function in place of the steerable guidewire, thereby eliminating the need for the steerable guidewire and reducing the number of insertion steps. Infusate (e.g., tPA) is then injected into the microcatheter 30, enters the support element 60 at its first helical section 60b, flows between the reduced diameter guidewire portion 50b and the support element section 60b and out of the support element at its second helical section 60d and into the balloon 70. Sufficient pressure is applied to the infusate to inflate the balloon 70 and cause the infusate to weep out of the pores 70e of the balloon and into the clot or into the walls of the blood vessel (not shown). When sufficient infusate has been introduced into the clot or vessel walls, the pressure is removed, the balloon 70 deflates, and the microcatheter 30 and guidewire subassembly 40 may be removed from the aspiration catheter 20. Alternatively, one or more subsequent processes of infusion and inflation can be carried out prior to removal of the microcatheter and guidewire. Suction may then be applied to the aspiration catheter 20 in order to remove the clot. The aspiration catheter 20 is then removed and the artery (not shown) is closed. The design of the microcatheter 30 and guidewire subassembly 40, which allows construction of the elements with very small outer diameters, permits the system to be effectively used in smaller vessels than permitted with other known devices.

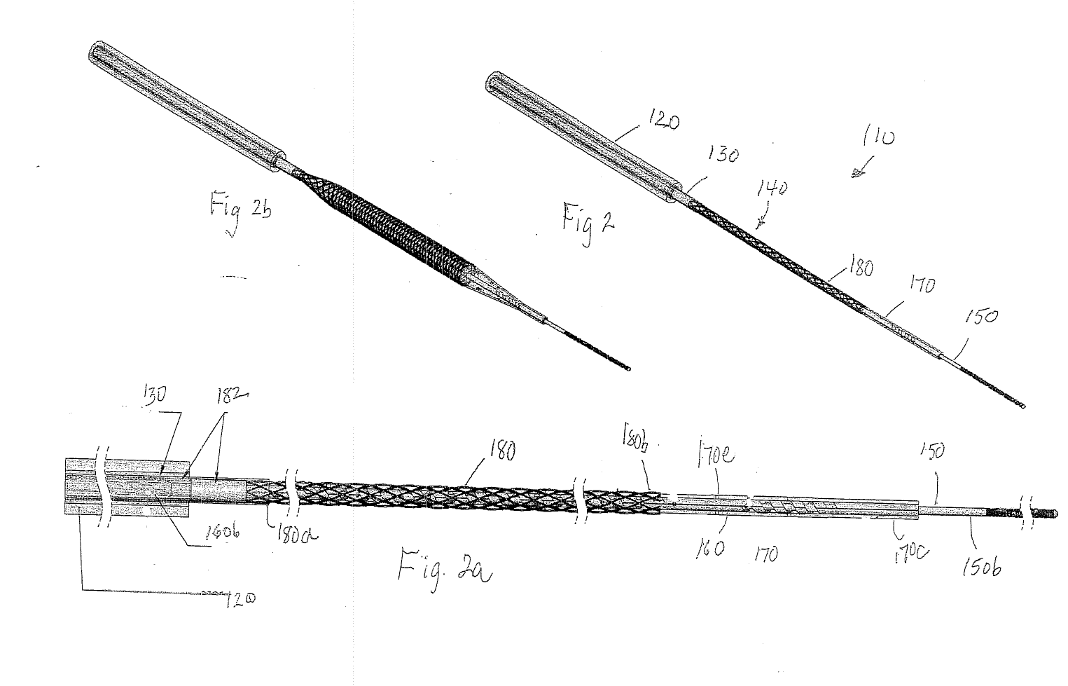

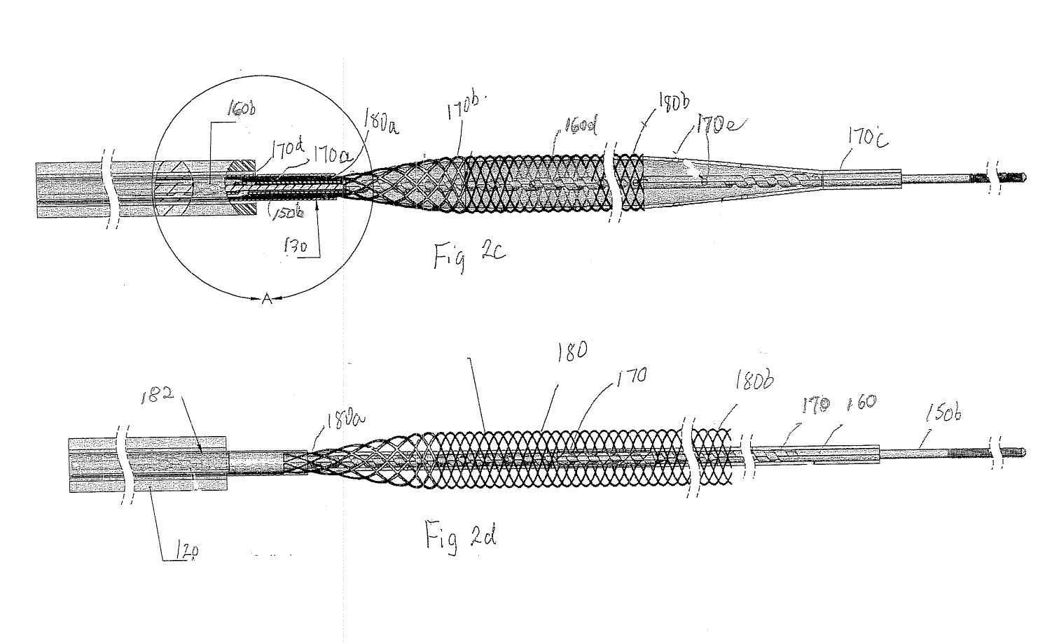

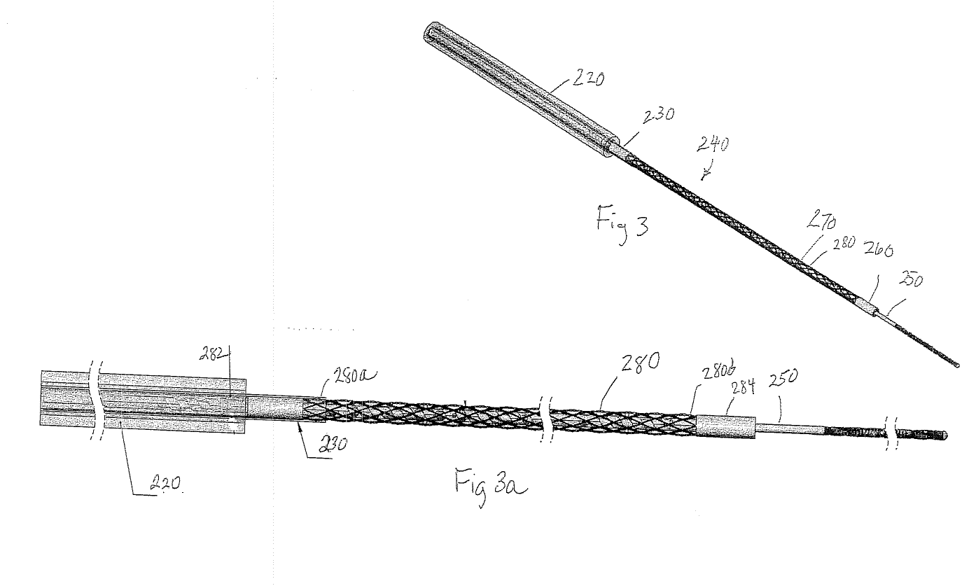

A second embodiment of the invention is seen in FIGS. 2 and 2a-2f. The second embodiment is similar in many respects to the first embodiment and is described in a manner where like parts are given like numbers which are one-hundred apart. Thus, system 110 includes an aspiration catheter 120, a microcatheter 130, and a guidewire subassembly 140, where the guidewire subassembly includes a guidewire 150, a support element 160, and a weeping or microjet balloon 170. All of these elements may be identical to, or substantially the same as their counterparts in the system 10 of FIGS. 1 and 1a-1e. System 110, however, further includes a balloon-deformable cage or stent 180. Cage 180 has a proximal end 180a which is optionally attached to a tubular cage tether 182 and a free distal end 180b preferably located proximal the distal end 170c of balloon 170. Where there is no cage tether, the proximal end 180a of the cage is affixed directly to the proximal end 170a of the balloon 170 (just distal the seal portion 170d) by gluing or affixing by other processes known in the art. Where there is a cage tether, the tubular cage tether 182 is glued or otherwise affixed to the proximal end 170a of the balloon 170.

As seen best in FIG. 2a, the cage 180 is chosen to have an inner diameter which either contacts the outer surface of the balloon or is just slightly larger than the outer surface diameter of the balloon when the cage (and balloon) is in an initial unexpanded position. As seen in FIGS. 2b and 2c, inflation of the balloon causes the middle portion 170b of the balloon to expand the cage to an expanded position. In addition, when the balloon is expanded, infusate may weep or jet out of the holes 170e located along the balloon. According to one aspect of the invention, the cage may be arranged so that it limits the ability of the balloon to expand beyond a certain diameter. This may be done by either designing the cage with a limited ability to expand, or by arranging the cage to provide a sufficient force when it reaches a particular diameter which would prevent the balloon from expanding. In technical terms, the resistive force (Fr) of the cage 180 is greater than or equal to the opening force of the balloon 170 for a given diameter (Fo). The balloon opening force will vary according to the number of and size of the infusate holes. The force limiting aspect of the cage can be broken down into two separate embodiments. If the material of the cage has a high tensile strength, e.g., spring steel, or is superelastic, e.g., Nitinol, the balloon will expand until Fr=Fo. The diameter will be a function of the respective forces (i.e. the cage and balloon design will have corresponding maximum diameters, e.g., 2 mm. When the balloon is deflated, the cage will return to a collapsed position. If the material of the cage is inelastic, e.g., annealed stainless steel, then when Fr=Fo and the diameter is achieved, the cage will remain in the expanded position even when the balloon is deflated, leaving a conduit for blood to flow. The cage can be removed by pulling the guidewire/microcatheter subassembly into the aspiration catheter, or retracting just the guidewire relative to the microcatheter.

As an alternative to a cage for controlling expansion of the balloon, the balloon may be constructed of a compliant material. The holes in the balloon may then function as a pressure relief; as the balloon expands, the holes get larger (in distinction from non-compliant balloons). As another alternative, a pressure relief can be provided within or coupled to the instrument to control and limit pressure. By way of example, an external pressure relief valve can be connected to a luer fitting on the hub of the microcatheter or to a touhy borst valve, which is then connected to the hub of the microcatheter. As yet another alternative, the infusion rate can be controlled by the use of a flow restrictor.

The guidewire/catheter system 110 may be used as follows. First, either the femoral or carotid artery (not shown) is punctured and a sheath (not shown) inserted. A steerable guidewire (not shown) is inserted into the sheath and steered until it crosses the clot of interest (not shown). The sheath optionally may then be removed, and the aspiration catheter 120 inserted through the puncture over the guidewire and up to just proximal the clot. The microcatheter 130 is then fed between the aspiration catheter 120 and the guidewire until it extends out of the aspiration catheter and into or through the clot. The steerable guidewire is then removed, and the guidewire subassembly 140, comprising the guidewire 150, attached support element 160, balloon 170, and cage 180, is inserted into the microcatheter 130 and snaked through the microcatheter until the balloon 170 is located in the clot (with the distal end 150b of the guidewire typically extending past the clot). Infusate (e.g., tPA) is then injected into the microcatheter 130, enters the support element 160 at its first helical section 160b, flows between the reduced diameter guidewire portion 150b and the support element section 160b and out of the support element at its second helical section 160d and into the balloon 170. Sufficient pressure is applied to the infusate to inflate the balloon 170 and cause the infusate to weep or jet out of the pores 170e of the balloon and into the clot or into the walls of the blood vessel (not shown) as well as expanding the cage 180 so that the cage presses against the walls of the blood vessel. When sufficient infusate has been introduced into the clot or vessel walls, the pressure is removed, the balloon 170 deflates, and if the cage 180 is biased toward a collapsed position, the cage collapses. The microcatheter 130 and guidewire subassembly 140 are then removed from the aspiration catheter 120. Suction may then be applied to the aspiration catheter 120 in order to remove the clot. The aspiration catheter 120 is then removed, the sheath (if present) is removed, and the artery (not shown) is closed. It is noted that if the cage 180 is not biased toward a collapsed position, when the balloon 170 deflates, the cage remain in an expanded position. Pulling the guidewire subassembly 140 including the cage proximally into the microcatheter 130 or the aspiration catheter 120, or pushing the microcatheter 130 forward relative to the cage 180 will cause the cage to collapse, whereupon, the microcatheter 130 and guidewire subassembly 140 may be removed from the aspiration catheter 120. Suction may then be applied as previously described, and then the catheter 120 may be removed and the artery closed.

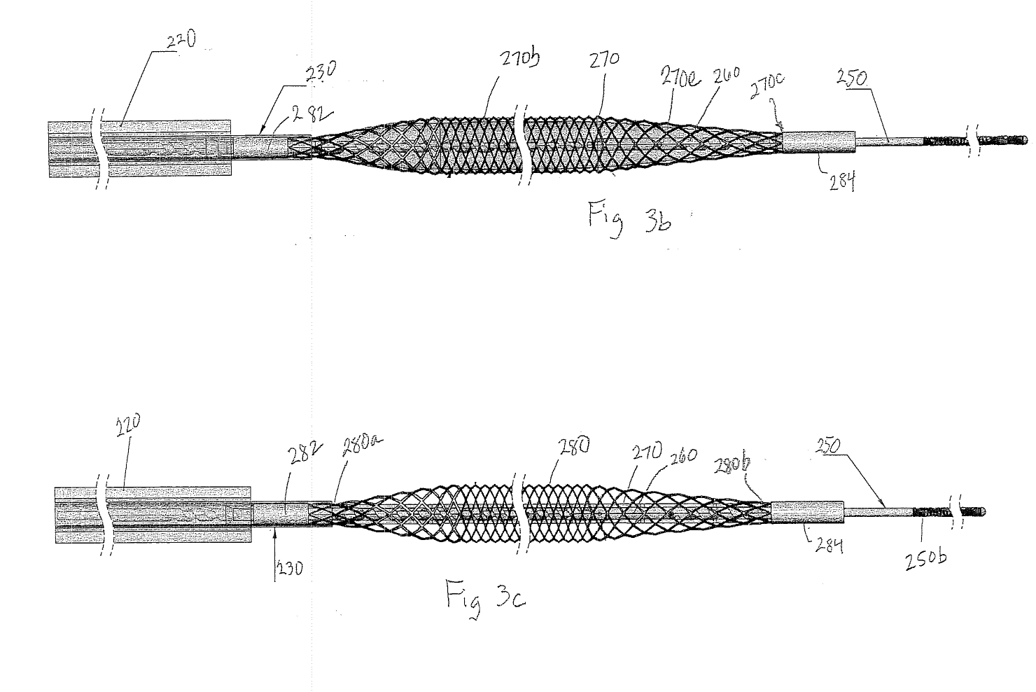

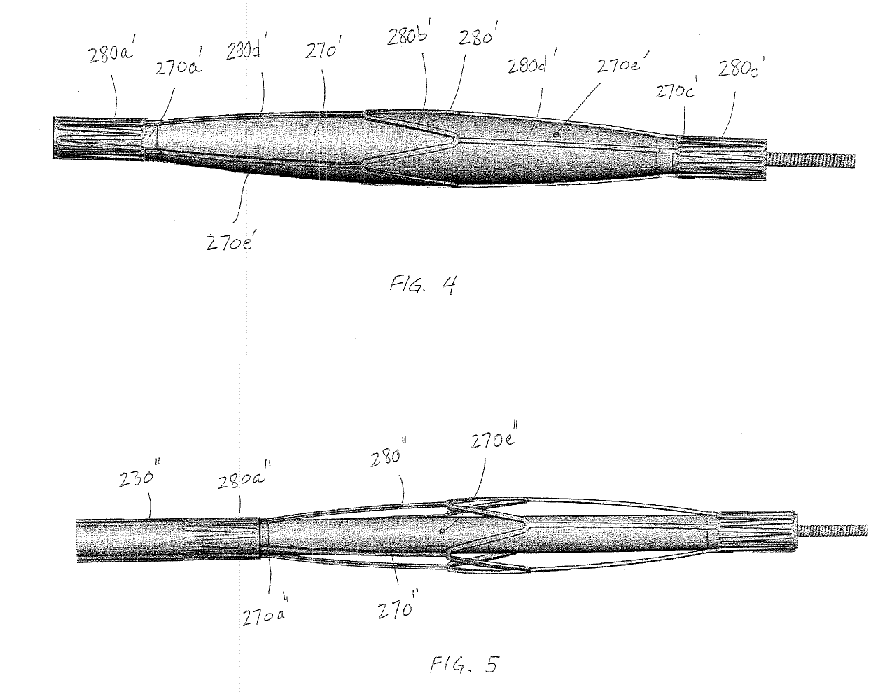

A third embodiment of the invention is seen in FIGS. 3 and 3a-3c. The third embodiment is similar in many respects to the second embodiment and is described in a manner where like parts are given like numbers which are one-hundred apart. Thus, system 210 includes an aspiration catheter 220, a microcatheter 230, and a guidewire subassembly 240, where the guidewire subassembly includes a guidewire 250, a support element 260, a weeping or jetting balloon 270, and a cage 280. All of these elements may be identical to, or substantially the same as their counterparts in the system 110 of FIGS. 2 and 2a-2d except that cage 280 has a distal end 280b which is affixed either to the distal end 260e of the support element 260, the distal end 270c of the balloon, or to the guidewire 250. Affixation of the distal end 280b of the cage 280 may be accomplished with the use of a second tubular cage tether 284 or by directly affixing the distal end of the cage to the balloon 270, support element 260 or to the guidewire 250. Similarly, and as in the second embodiment, the proximal end 280a of the cage 280 may likewise be affixed to the proximal end 270a of the balloon 270 either directly or via a tubular cage tether 282.

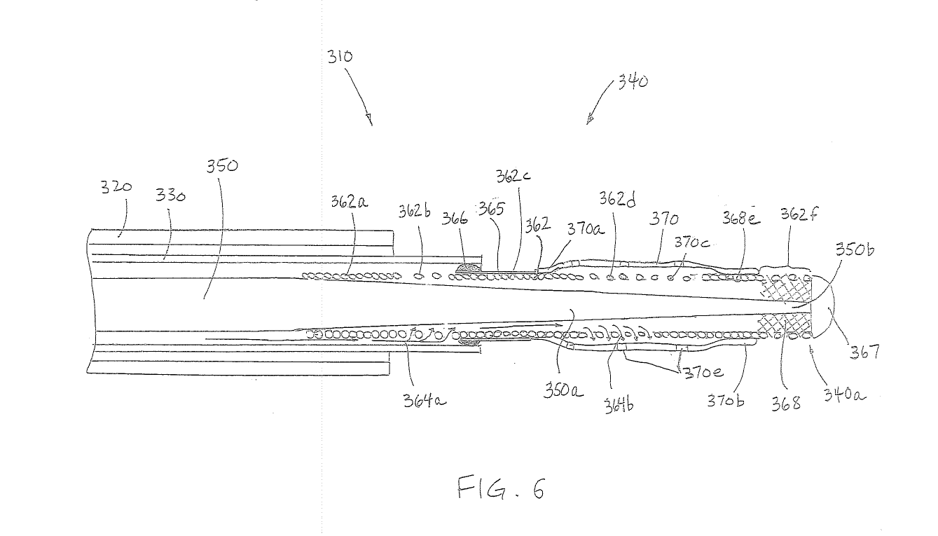

As seen best in FIG. 3a, the cage 280 is chosen to have an inner diameter which either contacts the outer surface of the balloon or is just slightly larger than the outer surface diameter of the balloon when the cage (and balloon) is in an initial unexpanded position. The cage 280 may be constructed of a braid of wires or other structural elements that extend from the proximal to distal ends of the balloon. Alternatively, as seen in FIG. 4, the cage 280′ may be constructed to include a central ring 280b′ formed by a series of Z-bends in a wire-form or from a laser-cut or stamp-cut form that is radially expansible. The central ring 280a′ is coupled to a proximal portion 270a′ and optionally a distal portion 270c′ of the balloon 270 with a plurality of longitudinally arranged struts 280d′ and non-expansible proximal and distal rings 280a′ and 280c′, which may also be formed from a series of Z-bends. Optionally additional radially expansible rings (not shown) may be provided to the cage 280′. As yet another modification of the design, as shown in FIG. 5, the proximal end 280a″ of the cage 280″ may be integrated with the distal end of the microcatheter 230″ (rather than coupled to the proximal end 270a″ of the balloon 270″, as previously described). In such a configuration, the cage essentially has a common diameter with the microcatheter. Also, in such a configuration, the distal end of the cage is not attached to the distal end of the balloon 270″. However, even though not attached to the balloon 270″, when the balloon 270″ is expanded, the middle portion of the cage will expand accordingly.

Referring back to FIG. 3b, inflation of the balloon causes the middle portion 270b of the balloon to expand the cage (all described designs) to an expanded position. In addition, when the balloon is expanded, infusate may weep or jet out of the holes 270e located along the balloon. Referring again to FIGS. 4 and 5, jetting is facilitated with fewer holes, such as the two holes 270e′ of balloon 270′, preferably longitudinally spaced along the length of the balloon one-third the balloon-length in from the proximal end and one-third the balloon-length in from the distal end of the balloon (FIG. 4), or one hole 270e″ of balloon 270″ (FIG. 5).

According to one aspect of the invention, the cage may be arranged so that it limits the ability of the balloon to expand beyond a certain diameter. This may be done by either designing the cage with a limited ability to expand, or by arranging the cage to provide a sufficient force when it reaches a particular diameter which would prevent the balloon from expanding. According to another aspect of the invention, the cage may be arranged so that it does not significantly impact the expansion of the balloon, and the cage 280 will expand to whatever diameter the balloon 270 (FIG. 3b), 270′ (FIG. 4), 270″ (FIG. 5) expands. According to another aspect of the invention, and as seen in FIG. 3c, the cage 280 may be arranged so that when the balloon 270 deflates after it has been inflated, the cage remains expanded. If the cage is arranged to remain expanded, movement of the microcatheter 230 distally relative to the cage will cause the cage to collapse inside the microcatheter or retraction of the guidewire/microcatheter assembly into the aspiration catheter will cause the cage to collapse (assuming the expanded diameter of the cage is larger than the inner diameter of the aspiration catheter). According to a further aspect of the invention, the cage 280 may be spring biased toward a closed position such that when the balloon is no longer being inflated by infusate, the cage 280 will return to a collapsed position. The guidewire/catheter system 210 may be used in the same manner as the guidewire/catheter system 110 of FIGS. 2 and 2a-2d.

As alternate to the above described arrangement, the support for the balloon is two discrete and longitudinally displaced sections. A first section includes a proximal portion attached to the guidewire, a helical portion extending from the proximal portion, and a first support portion extending from the helical portion. The second section is coupled to the guidewire, and the distal end of the balloon is coupled to the second section. The location and coupling of the second section is preferably the same as described above with respect to the guidewire 50, tubular support portion 60e, and the balloon 70 (FIGS. 1 and 1c). In this arrangement, no section of the balloon support, helical or otherwise, extends continuously through the balloon.

Turning now to FIG. 6, a fourth embodiment of the invention is seen. The fourth embodiment is similar in many respects to the first embodiment and is described in a manner where like parts are given like numbers. Thus, system 310 includes an aspiration catheter 320, a microcatheter 330, and a guidewire subassembly 340. The aspiration catheter and microcatheter may be identical to, or substantially the same as their counterparts in the system 10 of FIGS. 1 and 1a-1e. The guidewire subassembly 340 of system 310, however, is different including a guidewire core 350, a helical wound coil element 362, and a weeping or microjet balloon 370.

The guidewire core 350 is preferably constructed of a wire having a diameter of approximately 0.014 inches from its proximal end to a distal tapering diameter portion 350a. The tapering diameter portion 350a is preferably approximately 1 to 3.3 inches in length, and the guidewire core tapers down to approximately 0.003 inches at or adjacent its distal tip 350b.

The balloon 370 is made from a polymer preferably having a material thickness of approximately 0.002 to 0.008 inches. Infusate is permitted to flow between the tapering diameter portion 350a of the guidewire 350 and the helical coil element 362 and into the balloon 370 as described hereinafter.

The coil element 362 extends over the tapering diameter portion 350a of the guidewire. The coil element 362 is constructed of helically wound platinum/stainless steel or Nitinol wire, preferably having a wire diameter of approximately 0.003 inches. The coil element 360 includes (i) a tight pitch, closed wound first portion 362a preferably having a length of approximately 0.2 to 0.7 inches, (ii) a loose pitch, open wound second portion 362b preferably having a length of approximately 0.2 to 0.7 inches, (iii) a tight pitch, closed wound third portion 362c preferably having a length of approximately 0.2 to 0.7 inches, (iv) a loose pitch, open wound fourth portion 362d preferably having a length of approximately 0.2 to 0.7 inches, (v) a tight pitch, closed wound fifth portion 362e preferably having a length of approximately 0.2 to 0.5 inches, and (vi) a loose pitch, open wound sixth portion 362f preferably having a length of approximately 0.08 to 0.25 inches. The first portion 362a of the coil element is connected to the core wire 350 at or adjacent the proximal end of the tapering diameter portion 350a. The open wound second portion 362b of the coil element permits infusate within the microcatheter 330 to flow between the coil element and the tapering diameter portion 350a of the guidewire core 350 (as indicated by arrows 364a). The closed wound third portion 362c is coated with a polymeric thin layer 365, preferably approximately 0.001 to 0.003 inches in material thickness, that fluid seals the third portion 362c yet maintains the flexibility of the coil element 362. A ring seal 366, preferably formed as a bead of polymer on the proximal end of the third portion 362c, is in contact with the inner surface of the microcatheter and prevents infusate from exiting the distal end of the microcatheter 330. The proximal end 370a of the balloon 370 is bonded over the polymeric thin layer 365 or directly to the windings of the closed wound third portion 362c, and the distal end 370c of the balloon is bonded to the close wound fifth portion 362e. The open wound fourth portion 362d permits infusate within the coil element to flow out of the coil element 362 and into the surrounding balloon 370 (as shown by arrows 364b). The distal ends of the core wire 350 and coil element 362 are provided with a blunt atraumatic tip 367 that may be integrally formed with the core wire 350. A polymer 368 is injected into the open wound sixth portion 362f of the coil element to permanently fluid seal the distal tip 340a of the guidewire subassembly 340.

The guidewire/catheter system 310 may be used as follows. First, either the femoral or carotid artery (not shown) is punctured and a sheath (not shown) inserted. A steerable guidewire (not shown) is inserted into the sheath and steered until it crosses the clot of interest (not shown). The sheath may then be removed, and the aspiration catheter 320 is inserted through the puncture over the steerable guidewire and up to just proximal the clot. The microcatheter 330 is then fed between the aspiration catheter 320 and the guidewire until it extends out of the aspiration catheter and into or through the clot. The steerable guidewire is then removed, and the guidewire subassembly 340, comprising with the guidewire 350, coil element 362, and balloon 370, is inserted into the microcatheter 330 and snaked through the microcatheter until the balloon 370 is located in the clot (with the distal end 350c of the guidewire typically extending past the clot). Alternatively, the guidewire subassembly may be used initially to function in place of the steerable guidewire, thereby eliminating the need for the steerable guidewire and reducing the number of insertion steps. Infusate (e.g., tPA) is then injected into the microcatheter 330, enters the open wound second portion 362b of the coil element 362, flows between the tapered diameter portion of the core wire 350a and the coil element 362, and into the balloon 370. Sufficient pressure is applied to the infusate to inflate the balloon 370 and cause the infusate to weep out of the pores 370e of the balloon and into the clot or into the walls of the blood vessel (not shown). When sufficient infusate has been introduced into the clot or vessel walls, the pressure is removed, and the balloon 370 deflates. The microcatheter 330 and guidewire subassembly 340 are then removed from the aspiration catheter 320. Suction may then be applied to the aspiration catheter 320 in order to remove the clot. The aspiration catheter 320 is then removed, the sheath (if present) is removed, and the artery (not shown) is closed.

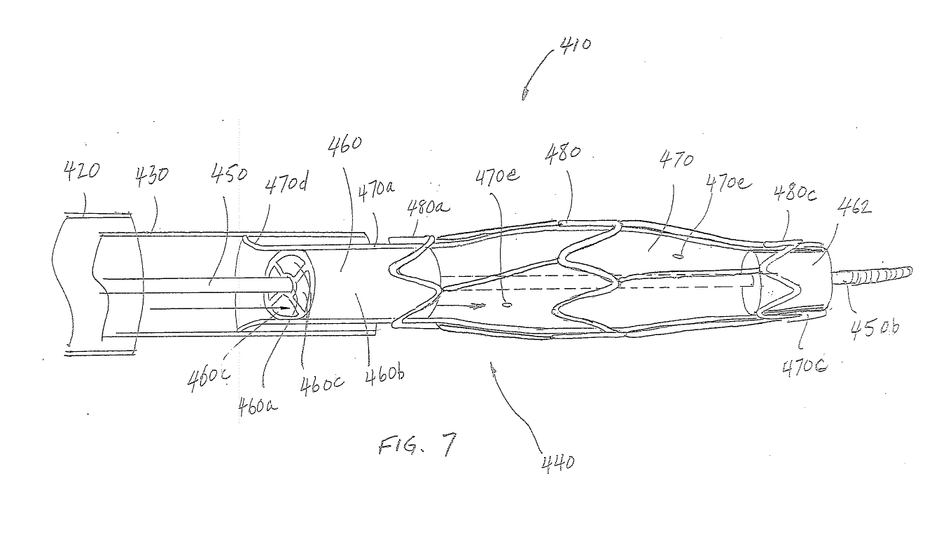

Referring to FIG. 7, a fifth embodiment of the invention is seen. The fifth embodiment is similar in many respects to the earlier embodiments and is described in a manner where like parts are given like numbers. Thus, system 410 includes an aspiration catheter 420, a microcatheter 430, and a guidewire subassembly 440. The aspiration catheter and microcatheter may be identical to, or substantially the same as their counterparts in the system 10 of FIGS. 1 and 1a-1e or system 310 of FIG. 6. The guidewire subassembly 440 of system 410, however, is different including a guidewire core 450, a hub 460, and a weeping or microjet balloon 470.

The guidewire core extends through the microcatheter 430 and through the balloon 470 of the subassembly 440. The guidewire core and balloon are preferably of any construction described in the earlier embodiments. However in distinction from the earlier embodiments, the core wire 450 extends through a hub 460 to which the proximal end of the balloon is affixed. The hub includes a central bore 460a through which the core wire 450 extends, an outer surface 460b which is in contact with the inner surface of the proximal end 470a of the balloon, and passageways 460c through which the infusate can flow from the microcatheter 430 to the interior of the balloon. The balloon includes a flared proximal opening 470d which contacts the inner surface of the microcatheter 430 to prevent infusate from leaking out of the micrcatheter between the microcatheter and the balloon. The distal end of the balloon 470c is provided about a distal support 462 which is fixedly mounted at the distal end 450e of the core wire. When infusate is forced through the microcatheter 430, it travels through the passageways 460c, inflates the balloon 470 and then is directed out of the balloon through holes 470e and into contact with the clot. As described in the above embodiments, a self-expandable or pressure-expandable cage 480 is optionally provided over the balloon and operates to limit expansion of the balloon and/or temporarily maintain patency through the vessel after the balloon is deflated. The proximal end of the cage is preferably coupled over the hub 460, and the distal end of the cage is preferably coupled over the distal support 462.

The guidewire/catheter system 410 may be used as follows. First, either the femoral or carotid artery (not shown) is punctured and a sheath (not shown) inserted. A steerable guidewire (not shown) is inserted into the sheath and steered until it crosses the clot of interest (not shown). The sheath may then be removed, and the aspiration catheter 420 is inserted through the puncture over the steerable guidewire and up to just proximal the clot. The microcatheter 430 is then fed between the aspiration catheter 420 and the guidewire until it extends out of the aspiration catheter and into or through the clot. The steerable guidewire is then removed, and the guidewire subassembly 440, comprising with the guidewire 450, hub 460 and balloon 470, is inserted into the microcatheter 430 and snaked through the microcatheter until the balloon 470 is located in the clot (with the distal end 450b of the guidewire typically extending past the clot). Alternatively, the guidewire subassembly may be used initially to function in place of the steerable guidewire, thereby eliminating the need for the steerable guidewire and reducing the number of insertion steps. Infusate (e.g., tPA), preferably in combination with a fluoroscopic contrast agent, is then injected into the microcatheter 430, enters through the passageways 460c in the hub 460, and into the balloon 470. Sufficient pressure is applied to the infusate to inflate the balloon 470 and cause the infusate to weep or jet out of the holes 470e of the balloon 470 and into the clot or into the walls of the blood vessel (not shown). When a contrast agent is used, expansion of the balloon as well as the flow of the infusate out of the balloon is visualized with standard fluoroscopic equipment. As such, visualization of recannulization can be viewed in real-time. When sufficient infusate has been introduced into the clot or vessel walls, the pressure is removed, and the balloon 470 deflates. The cage, if provided, may then automatically collapse, or be moved against the distal end of one of the microcatheter 430 or aspiration catheter 420 to force its collapse. The microcatheter 430 and guidewire subassembly 440 are then removed from the aspiration catheter 420. Suction may then be applied to the aspiration catheter 420 in order to remove the clot. The aspiration catheter 420 is then removed, the sheath (if present) is removed, and the artery (not shown) is closed.

In an experiment using rabbits with induced blood clots in vessels of similar size to the human middle cerebral artery, a device as described with reference to FIG. 6 was shown to be effective in (1) delivering tPA directly within an occluding thrombus, (2) creating flow in the occluded vessel, and (3) resulting in an acceptable level of intimal/medial disruption. In the experiment, the device of the invention was delivered through a microcatheter, and the balloon was positioned within the clot. An infusion of the tPA Alteplase was mixed with a contrast agent in a 1:1 ratio and was infused from the distal to the proximal end of the clot. Multiple dilatations with the balloon were carried out with the inflation pressure monitored and kept between 760-1520 Torr. For comparison purposes, an angioplasty balloon was also positioned within a clot and multiple inflations were carried out from the distal to the proximal end, and a delivery microcatheter was positioned within a clot and the Alteplase dose diluted with saline in a 1:3 ratio which was infused from the distal to the proximal end of the clot. After the experiment it was concluded that with the device of the invention, Alteplase was able to be delivered directly within the occluding thrombus and achieve recanalization early and with a reduced thrombolytic dose in comparison with standard thrombolytic infusion techniques (delivery microcatheter) and mechanical disruption (balloon angioplasty) alone.

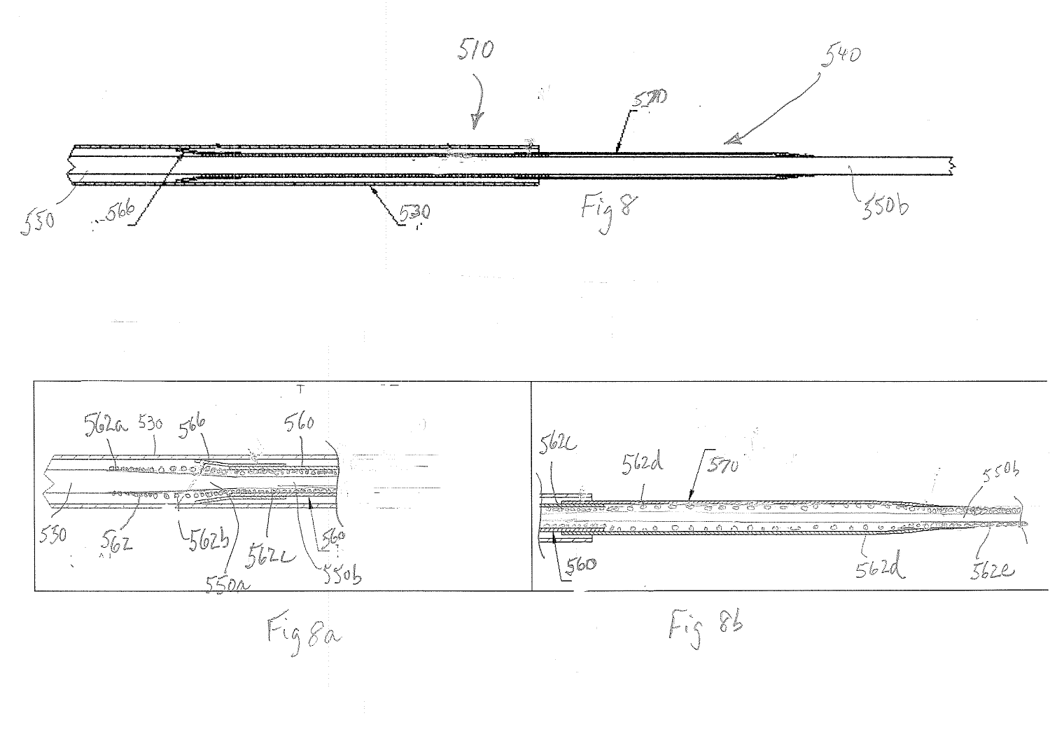

A sixth embodiment of the invention is seen in FIGS. 8, 8a and 8b. The sixth embodiment is similar in many respects to the earlier embodiments and is described in a manner where like parts are given like numbers. Thus, system 510 includes an aspiration catheter (not shown), a microcatheter 530, and a guidewire subassembly 540. The aspiration catheter and microcatheter may be identical to, or substantially the same as their counterparts in the system 10 of FIGS. 1 and 1a-1e or system 310 of FIG. 6. The guidewire subassembly 540 of system 510, however, is different including a guidewire core 550, a helical wound coil element 562, a weeping or microjet balloon 570, an intermediate tube 560, and a seal 566.

As seen best in FIGS. 8a and 8b, the coil element 562 extends over the tapering diameter portion 550a of the guidewire. The coil element 562 is constructed of helically wound platinum/stainless steel or Nitinol wire. The coil element 560 includes (i) a tight pitch, closed wound first portion 562a (ii) a loose pitch, open wound second portion 562b, (iii) a tight pitch, closed wound third portion 562c, (iv) a loose pitch, open wound fourth portion 562d, and (v) a tight pitch, closed wound fifth portion 562e. A distal loose pitch, open wound sixth portion may be provided if desired. The first portion 562a of the coil element is connected to the core wire 550 at or adjacent the proximal end of the tapering diameter portion 550a. The open wound second portion 562b of the coil element permits infusate within the microcatheter 530 to flow between the coil element and the tapering diameter portion 550a of the guidewire core 550 (as in the arrangement of FIG. 6). The closed wound third portion 562c is coupled to the intermediate tube 560, and is of a diameter that permits the infusate to continue to flow between it and a reduced diameter portion 550b of the guidewire core 550. As seen best in FIG. 8a, the seal 566 is attached to the proximal end of the intermediate tube 560 and flares outwardly and over the coil element 562 as it extends proximally into contact with the inside of the microcatheter 530; while as seen best in FIG. 8b, the proximal end of the balloon 570 is attached to the distal end of the intermediate tube 560, and the distal end of the balloon is attached to the fifth wound tightly wound portion 562e of the coil. Tightly wound portion 562e of the coil is in turn attached to the reduced diameter portion 550b of the guidewire, so that infusate cannot flow past the distal end of the balloon. Instead, the fourth loosely wound portion 562d of the coil is located inside the balloon 570 and permits infusate which is flowing between the coil 52 and the guidewire 550 to flow outwardly in order to inflate the balloon 570 and, if the balloon is provided with pores, to weep or jet out of the pores of the balloon.

Optionally, the seal 566 may be made of polyurethane, the intermediate tube 560 made of polyolefin, and the balloon 570 made of a biocompatible elastomer such as ChronoPrene (a trademark of AdvanSource Biomaterials Corp. of Massachusetts). The seal and intermediate tube can be joined by “welding” them together using heat and/or pressure. Likewise, the intermediate tube and balloon can be joined by “welding” them together using heat and/or pressure. In this manner, an effectively single element of different stiffnesses and functions is generated, with the intermediate tube being stiffer than the balloon and seal. Of course, other materials and connecting methods could be utilized.

The sixth embodiment of the guidewire/catheter system may be used in much the same manner as one or more of the previously described embodiments.

There have been described and illustrated herein several embodiments of a system and a method of treating a blood clot from the intracranial vasculature of a patient. While particular embodiments of the invention have been described, it is not intended that the invention be limited thereto, as it is intended that the invention be as broad in scope as the art will allow and that the specification be read likewise. It is noted that the word “approximately” used herein means the range within (+) or (−) 20 percent of the value which follows the word “approximately”. While particular preferred diameters and sizes of catheters, elongate members, and balloons have been disclosed, it will be appreciated that minor modifications to the shapes and sizes of the catheters, elongate members, and balloons which also accomplish the functionality of the system may be utilized. It will therefore be appreciated by those skilled in the art that yet other modifications could be made to the provided invention without deviating from its spirit and scope as claimed.

What is claimed is:

1. A system for use in a blood vessel having a blood clot and in conjunction with an infusate, the system comprising:
a microcatheter having an inner surface and a distal end, and said microcatheter providing a lumen through which the infusate can flow; and
a guidewire subassembly extending through and beyond said distal end of said microcatheter, said guidewire subassembly comprised of a guidewire core, a support element coupled to and extending around a portion of said guidewire core, and a balloon coupled to and extending around said support element, wherein said balloon includes a proximal seal in contact with said inner surface of said microcatheter and at least one hole along a length of said balloon, said support element includes
(i) a proximal portion attached to said guidewire core,
(ii) a first helical portion extending from said proximal portion and which extends around said guidewire core wherein said infusate can flow into said support element at said helical portion and between said support element and said guidewire core,
(iii) a first support section extending from said first helical portion, a proximal portion of said balloon attached to said first support section, and
(iv) a second helical portion distally extending from said first support portion through which said infusate can flow out of said support element and into said balloon in order to inflate said balloon,
wherein said infusate can flow between said support element and said guidewire core.

2. A system according to claim 1, further comprising:
an aspiration catheter attachable to a source of suction, said microcatheter extending through said aspiration catheter.

3. A system according to claim 1, wherein:
said guidewire core has a first diameter at a first location where said support element is attached to said guidewire core and a second diameter smaller than said first diameter at a second location distal said first location where said support element extends around said guidewire core.

4. A system according to claim 3, wherein:
said support element includes a distal portion attached to said guidewire core and including a second support section to which a distal portion of said balloon is attached.

5. A system according to claim 1, wherein:
said second helical section extends from said first support section to said second support section.

6. A system according to claim 1, wherein:
said guidewire core has a decreased diameter portion, and the infusate flows between said decreased diameter portion and said support element.

7. A system according to claim 1, further comprising:
a cage element coupled around said balloon.

8. A system according to claim 7, wherein:
said cage element has a proximal end coupled to a proximal end of said balloon.

9. A system according to claim 7, wherein:
said cage element has a proximal end coupled to a distal end of said microcatheter.

10. A system according to claim 7, wherein:
said cage element has a free distal end.

11. A system according to claim 7, wherein:
said cage element has a distal end coupled to one of a distal end of said balloon, a distal end of said support element, and said guidewire core.

12. A system according to claim 7, wherein:
said cage is adapted to limit inflation of said balloon.

13. A system according to claim 12, wherein:
said cage is biased towards a collapsed position.

14. A system for use in a blood vessel having a blood clot and in conjunction with an infusate, the system comprising:
a microcatheter having an inner surface and a distal end, and said microcatheter providing a lumen through which the infusate can flow; and
a guidewire subassembly extending through and beyond said distal end of said microcatheter, said guidewire subassembly comprised of a guidewire core, a helically wound coil element coupled to and extending around a portion of said guidewire core, and a balloon coupled to and extending around said coil element, wherein said balloon includes or is coupled to a proximal seal in contact with said inner surface of said microcatheter and at least one hole along a length of said balloon, said coil element includes,
(i) a tight pitch, closed wound first portion coupled to said guidewire core,
(ii) a loose pitch, open wound second portion extending from said first portion and through which the infusate can flow from said microcatheter to a flow path between said coil element and said guidewire core,
(iii) a tight pitch, closed wound third portion extending from said second portion and to which a proximal end of said balloon is coupled,
(iv) a loose pitch, open wound fourth portion extending from said third portion and through which the infusate can flow into said balloon to inflate said balloon and out of said at least one hole, and
(v) a tight pitch, closed wound fifth portion extending from said fourth portion and to which a distal end of said balloon is coupled.

15. A system according to claim 14, further comprising:
an aspiration catheter attachable to a source of suction, said microcatheter extending through said aspiration catheter.

16. A system according to claim 14, wherein:
said coil element further includes an open wound sixth portion extending from said fifth portion, and a polymer is disposed within said sixth portion to seal a distal end of said guidewire subassembly.

17. A system according to claim 14, wherein:
said guidewire core has a reduced diameter portion, and said coil element extends over said reduced diameter portion such that a path for infusate flow is provided between said reduced diameter portion of said guidewire core and said coil element.

18. A system according to claim 14, wherein
said third portion of said coil element is coated with a thin polymeric layer to that fluid seals said third portion.

19. A system according to claim 14, wherein:
said guidewire subassembly includes a tube having a proximal end to which said seal is connected and a distal end to which said balloon is connected.

20. A system according to claim 20, wherein:
said seal is formed of a first polymeric material, said tube is formed of a second polymeric material different than said first polymeric material, and said balloon is formed of a third polymeric material different than said first and second polymeric materials.

21. A system according to claim 14, further comprising:
a ring about said coil element and in contact with said inner surface of said microcatheter, said ring preventing the infusate from exiting said distal end of said microcatheter.

22. A system for use in a blood vessel having a blood clot and in conjunction with an infusate, the system comprising:
a microcatheter having an inner surface and a distal end, and said microcatheter providing a lumen through which the infusate can flow; and
a guidewire subassembly extending through and beyond said distal end of said microcatheter, said guidewire subassembly comprised of a guidewire core, a support means coupled to and extending around a portion of said guidewire core, and a balloon coupled to and extending around said support means, wherein said balloon includes a proximal seal in contact with said inner surface of said microcatheter and at least one hole along a length of said balloon, said support means includes
(i) a proximal portion attached to said guidewire core,
(ii) a first helical portion extending from said proximal portion and which extends around said guidewire core wherein said infusate can flow into said support element at said helical portion and between said support element and said guidewire core,
(iii) a first support section extending from said first helical portion, a proximal portion of said balloon attached to said first support section, and
(iv) a second support section longitudinally spaced apart from and distinct from said proximal portion, said first helical portion and said first support section, said second support section attached to said guidewire core, and said distal end of said balloon coupled to said second support section,
wherein said infusate can flow in a spaced defined between said first helical portion and said first support section of said support means and said guidewire core.

23. A system for use in a blood vessel having a blood clot and in conjunction with an infusate, the system comprising:
a microcatheter having an inner surface and a distal end, and said microcatheter providing a lumen through which the infusate can flow; and
a guidewire subassembly extending through and beyond said distal end of said microcatheter, said guidewire subassembly comprised of,
(i) a guidewire core,
(ii) a support element coupled to and extending around a portion of said guidewire core, said support element defining a helical opening along a portion of its length opening into a fluid flow path defined between said guidewire core and said support element,
(iii) a balloon coupled to and extending around said support element, at least one hole along a length of said balloon, said fluid flow path in communication with said at least one hole of said balloon, and
(iv) means for ensuring that infusate within said microcatheter is directed into said blood vessel only through said at least one hole,
wherein infusate can flow through said lumen of said microcatheter into said helical opening and along said fluid flow path.

24. A system according to claim 23, wherein:
said microcatheter and said guidewire subassembly are longitudinally displaceable relative to each other.

25. A system for use in a blood vessel having a blood clot and in conjunction with an infusate, the system comprising:
a microcatheter having an inner surface and a distal end, and said microcatheter providing a lumen through which the infusate can flow; and
a guidewire subassembly extending through and beyond said distal end of said microcatheter, said guidewire subassembly comprised of,
(i) a guidewire core,
(ii) a support element coupled to and extending around a portion of said guidewire core, said support element defining a fluid flow passage,
(iii) a balloon coupled to and extending around said support element, said balloon having at least one hole situated along a length of said balloon to permit infusate to flow out of said balloon to a space exterior of said balloon,
wherein said lumen of said microcatheter and said at least one hole of said balloon are in fluid communication through said fluid flow passage of said support element, and
(iv) means for ensuring that infusate within said microcatheter is directed into said blood vessel only through said at least one hole,
wherein infusate can flow through said lumen of said microcatheter, through the fluid flow passage of said support element to inflate said balloon, and out of said at least one hole.

26. A system according to claim 25, wherein:
said support element is a hub, and said fluid flow passage extends straight through said hub.

27. A system according to claim 25, wherein:
said balloon includes a plurality of holes.

28. A system according to claim 25, wherein:
said microcatheter and said guidewire subassembly are longitudinally displaceable relative to each other.

29. A system according to claim 25, further comprising:
a cage element coupled around said balloon.

30. A system according to claim 29, wherein:
said cage element has a proximal end coupled to a proximal end of said balloon.

31. A system according to claim 29, wherein:
said cage element has a proximal end coupled to a distal end of said microcatheter.

32. A system according to claim 29, wherein:
said cage element has a free distal end.

33. A system according to claim 29, wherein:
said cage element has a distal end coupled to one of a distal end of said balloon, said support element, a discrete second support element, and said guidewire core.

34. A system according to claim 29, wherein:
said cage is adapted to limit inflation of said balloon.

35. A method for treating a blood clot in the intracranial vasculature of a patient, comprising:
a) incising the patient to form an incision;
b) distally advancing a system through the incision to a blood clot, the system comprising,
a guidewire subassembly extending through and beyond said distal end of said microcatheter, said guidewire subassembly comprised of,
(i) a guidewire core,
(ii) an element coupled to and extending around a portion of said guidewire core, said element at least partially defining a fluid flow passage,
(iii) a balloon coupled to and extending around said element, said balloon having an interior and at least one hole situated along a length of said balloon to permit flow out of said balloon and into the intracranial vasculature,
wherein said lumen of said microcatheter and said at least one hole of said balloon are in fluid communication through said fluid flow passage of said support element, and
(iv) means for ensuring that infusate within said microcatheter is directed into said blood vessel only through said at least one hole, wherein
said system is advanced such that said balloon is situated within the blood clot; and
c) infusing a blood clot dissolving infusate into said lumen of said microcatheter, through said fluid flow passage, into said balloon to inflate said balloon, and out of said at least one hole to thereby contact the infusate against the blood clot.

36. A method according to claim 35, further comprising:
repeatedly expanding and contracting said balloon within the cranial vasculature.

37. A method according to claim 35, further comprising:
aspirating the dissolved blood clot through an aspiration catheter.

38. A method according to claim 37, wherein:
said distally advancing step includes advancing the system through the aspiration catheter to the blood clot.

39. A method according to claim 35, further comprising:
longitudinally displacing one of said guidewire subassembly and said microcatheter relative to each other.

40. A method according to claim 35, wherein:
said guidewire subassembly includes a cage extending about said balloon, and when said cage is in an expanded position, said longitudinally displacing causes said cage to move into a relatively collapsed position.

41. A method according to claim 35, further comprising:
viewing recannulization of said vasculature in real-time when the infusate contacts the clot, wherein said infusate includes a contrast agent that is visible under fluoroscopy.

42. A method according to claim 35, wherein:
said infusing includes supplying sufficient pressure to said infusate to cause said infusate to jet out of said at least one hole in said balloon.

43. A method according to claim 35, wherein:
said infusate flows in a helical fluid flow path through said fluid flow passage.

44. A system for use in a blood vessel having a blood clot and in conjunction with an infusate, the system comprising:
a microcatheter having an inner surface and a distal end, and said microcatheter providing a lumen through which the infusate can flow; and
a guidewire subassembly extending through and beyond said distal end of said microcatheter, said guidewire subassembly comprised of a guidewire core, an element coupled to and extending around a portion of said guidewire core, and a balloon coupled to and extending around said element, wherein said balloon includes or is coupled to a proximal seal in contact with said inner surface of said microcatheter and at least one hole along a length of said balloon, wherein said infusate can flow between said element and said guidewire core and into said balloon.
